(19) United States
(12) Patent Application Publication
(10) Pub. No.: US 2022/0057390 A1
(43) Pub. Date: Feb. 24, 2022

(54) SALIVA TESTING KIT USING NANO CARBON IMMUNOCHROMATOGRAPHY

(71) Applicant: BiopolariX Scientific, LLC, Richardson, TX (US)

(72) Inventors: Xiaolong Hu, Shanghai (CN); Hong Zheng, Plano, TX (US); Fahmi Nogura, Hickoryhills, IL (US)

(21) Appl. No.: 17/407,735

(22) Filed: Aug. 20, 2021

Related U.S. Application Data
(60) Provisional application No. 63/068,014, filed on Aug. 20, 2020.

(51) Int. Cl. G01N 33/543; G01N 33/569; G01N 33/58

(52) U.S. Cl. CPC G01N 33/54388; G01N 33/56983; G01N 33/587; G01N 2800/26; G01N 2470/06; G01N 2333/165; G01N 2469/10

(57) ABSTRACT

Biological assay systems, methods, and devices for detecting the presence of the virus responsible for COVID-19 (sars-cov-2) in the saliva of an individual. The systems, methods, and devices utilize immunoassay technology for detecting the presence of antigen in a sample, such as the virus responsible for COVID-19 in the saliva of an individual. The immunoassay technology in accordance with embodiments of the systems, methods, and devices use nano-carbon, or carbon nanoparticles attached to biorecognition/detector molecules, such as antibodies.

### CROSS REFERENCE TO RELATED APPLICATIONS

In accordance with 37 C.F.R. 1.76, a claim of priority is included in an Application Data Sheet filed concurrently herewith. Accordingly, the present invention claims priority to U.S. Provisional Patent Application No. 63/068,014, entitled “Saliva Testing Kit Using Nano Carbon Immunochromatography”, filed on Aug. 20, 2020. The contents of the above referenced application are incorporated herein by reference in their entirety.

### FIELD OF THE INVENTION

The present invention relates to biological assay systems, methods, and devices, to biological assay systems, methods, and devices for detecting disease; and more particularly, to biological assay systems, methods, and devices for detecting the presence of the virus responsible for COVID-19 in the saliva of an individual.

### BACKGROUND OF THE INVENTION

The Coronavirus pandemic, which began in late 2019, continues to cause disease and death across the globe almost two years after first being identified as a public health emergency. With infection rates still on the rise and areas having multiple waves of infection, the need for detection remains high. In addition, at least for the near future, it appears that society must learn to live with the disease rather than eradicate it. Accordingly, to manage the outbreaks, properly diagnosing individuals as COVID-19 positive or negative is critical. For those individuals that test COVID-19 positive, they can immediately take measures, such as quarantine, wearing masks, or other medical interventions that would prevent spreading of the virus. For those individuals that test COVID-19 negative, they can go about their daily lives knowing they are not infected and will not be a transmitter. To have an ultimate impact, the COVID-19 test should be reliable and easy to administer. Results should be obtained quickly and should not require the user to obtain results from an outside provider, i.e. have to send the results away for processing or require an independent lab or physician to report the results.

Numerous technologies for detecting disease are known. Immunoassay technology uses the affinity of antigen and antibody to detect the substance in the sample. The structure, size and surface area of carbon nanoparticles are suitable for the adsorption or coupling of biomolecules, including protein molecules, enzymes, and DNA. These molecules are connected to the surface of carbon nanoparticles and can be used to prepare highly sensitive sensors. The biological assay systems, methods, and devices in accordance with embodiments of the invention use nano-carbon labeling technology applied to immunochromatographic detection to improve the sensitivity of immunochromatographic detection.

Compared with colloidal gold particles, carbon nanoparticles have higher sensitivity and stability. In 2008, Wei Qiuyan and others, Qiuyan et al., A Novel Carbon Nanoparticle Probe-Based Ultrasensitive Lateral Flow Assay For Rapid Detection Of Ebola Virus. Chin J. Biotech, 2018, 334(12); 2025-2034, labeled rabbit polyclonal antibodies against Ebola virus (EBOV) matrix protein VP40 with carbon nanoparticles, and assembled a colloidal carbon lateral flow immunochromatographic strip that can detect Ebola virus within 15 minutes. The detection limit of the colloidal carbon strip for detecting inactivated EBOV was 100 ng/ml (equivalent to 10⁸ copies/ml), which was much better than that of the colloidal gold strip (10 μg/ml, equivalent to 108 copies/ml). In 2018, Chen Zonglun et al. studied the aflatoxin immunochromatographic detection method. The naked eye detection sensitivity of colloidal gold detection card was 0.2 ng/ml, and that of colloidal carbon detection was 0.1 ng/ml.

In 2008, Che Hongli et al. established a colloidal strip immune-diagnosis method for rapid detection of Schistosoma japonicum antibody in serum. The method is simple and rapid, with high sensitivity and specificity, which is suitable for field diagnosis of schistosomiasis. In 2017, Shanghai Venture Biotechnology, Co. Ltd cooperated with Shanghai Public Security Bureau to detect methamphetamine in human samples by carbon nanotube technology, with a detection limit of 62.5 ng/ml and colloidal gold of 1000 ng/ml, with detection sensitivity increased by more than 10 times.

### SUMMARY OF THE INVENTION

Embodiments of the present invention relate to systems, methods, and devices for detecting disease. In an illustrative example, the systems, methods, and devices utilize immunoassay technology for detecting the presence of antigen in a sample, such as the virus (Sars-Cov-2 Virus) responsible for COVID-19 in the saliva of an individual. Immunoassay technology uses the affinity of antigen and antibody to detect the substance in the sample. The immunoassay technology in accordance with embodiments of the systems, methods, and devices of the present invention use nano-carbon, or carbon nanoparticles secured to biorecognition/detector molecules, such as antibodies. The structure, size and surface area of the carbon nanoparticles are suitable for the adsorption or coupling of biomolecules, including protein molecules, enzymes, and DNA. These molecules are connected to the surface of carbon nanoparticles and can be used to prepare highly sensitive sensors. In the embodiments of the systems, methods, and devices, nano-carbon labeling technology is applied to immunochromatographic detection, significantly improving the sensitivity of immunochromatographic detection.

The surface characteristics of carbon nanoparticles make it possible to modify the surface of carbon nanoparticles with active groups, such as amino acid group, carboxyl group and thiol group. These active groups can be covalently coupled with antibody, which makes them have the ability to recognize the detected substances, and can be used for medical diagnosis and biological molecular recognition. The most common applications of carbon nanotube sensing systems are DNA, chemical and immune sensors. The technology of antibody labeling carbon nanoparticles includes two methods: passive adsorption and covalent coupling. Passive adsorption is completed by electrostatic adsorption of antibody by nano-carbon. Covalent coupling is the combination of active groups on the surface of nano-carbon and covalent bond of antibody. Both methods are suitable for immunoassay. At present, the surface functionalized carboxyl group technology of carbon nanoparticles has been mature, which can be applied in this project.

The structure, size and surface area of carbon nanoparticles are suitable for the adsorption or coupling of biomolecules, including protein molecules, enzymes, and DNA. These molecules are connected to the surface of carbon nanoparticles and can be used to prepare highly sensitive sensors. In this project, nano-carbon labeling technology is applied to immunochromatographic detection, which can significantly improve the sensitivity of immunochromatographic detection.

Accordingly, it is a primary objective of the invention to provide systems, methods, and devices for detecting disease.

It is a further objective of the invention to provide systems, methods, and devices which utilize immunoassay technology for detecting the presence of antigen in a sample.

It is yet another objective of the invention to provide systems, methods, and devices which utilize immunoassay technology for detecting the presence of the virus responsible for COVID-19 in an individual.

It is a still further objective of the invention to provide systems, methods, and devices which utilize immunoassay technology for detecting the presence of the virus responsible for COVID-19 in a saliva sample of an individual.

It is a further objective of the invention to provide systems, methods, and devices which utilize immunoassay technology having carbon nanoparticles for detecting the presence of antigen in a sample.

It is yet another objective of the invention to provide systems, methods, and devices which utilize immunoassay technology having carbon nanoparticles for detecting the presence of the virus responsible for COVID-19 in a saliva sample of an individual.

It is a still further objective of the invention to provide systems, methods, and devices for rapid detection of the COVID-19 virus.

It is a further objective of the invention to provide systems, methods, and devices for rapid detection of the COVID-19 virus without the need for special instruments.

It is yet another objective of the invention to provide systems, methods, and devices for rapid detection of the COVID-19 virus without the need for special or medical professionals.

Other objects and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE FIGURES

FIG. 1 is an illustrative embodiment of a nano-carbon immunochromatography device;

FIG. 2A is an illustrative embodiment of a nano-carbon immunochromatography strip used in the nano-carbon immunochromatography device;

FIG. 2B illustrates the nano-carbon immunochromatography strip shown in FIG. 2A, with the sample/analyte detection antibodies and the primary and secondary antibodies;

FIG. 3 is an illustrative embodiment of a nano-carbon immunochromatography strip used in the nano-carbon immunochromatography device;

FIG. 4 illustrates one embodiment of using the nano-carbon immunochromatography device; and

FIG. 5 illustrates an alternative embodiment of using the nano-carbon immunochromatography device.

### DETAILED DESCRIPTION OF THE INVENTION

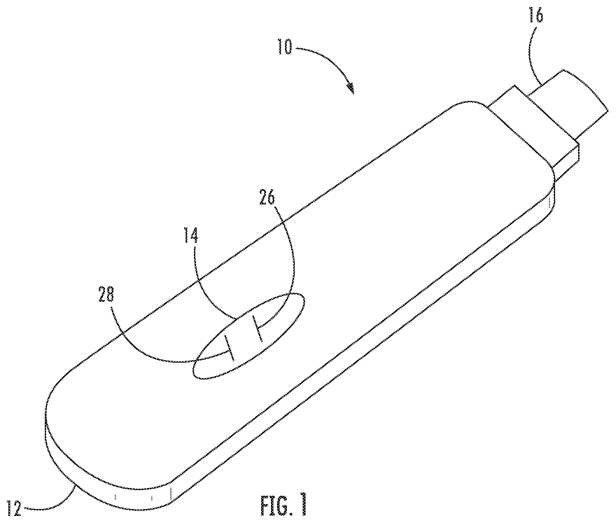

Referring to FIG. 1, an illustrative embodiment of an immunochromatography device having carbon nanoparticles, referred to generally as nano-carbon immunochromatography device 10, is shown. The nano-carbon immunochromatography device 10 uses the affinity of an antigen and an antibody to detect the substance in the sample. The nano-carbon immunochromatography device 10 includes a housing unit 12 with a viewing or indicating window 14 and an immunochromatography test strip 16. The viewing or indicating window 14 is sized and shaped to allow a user to visualize an indicator which designates the detection, or lack of detection, of a substance in a sample to be tested. Although not illustrated, the nano-carbon immunochromatography device 10 may include a sample deposit reservoir to allow the sample to be analyzed to be dropped or placed therein.

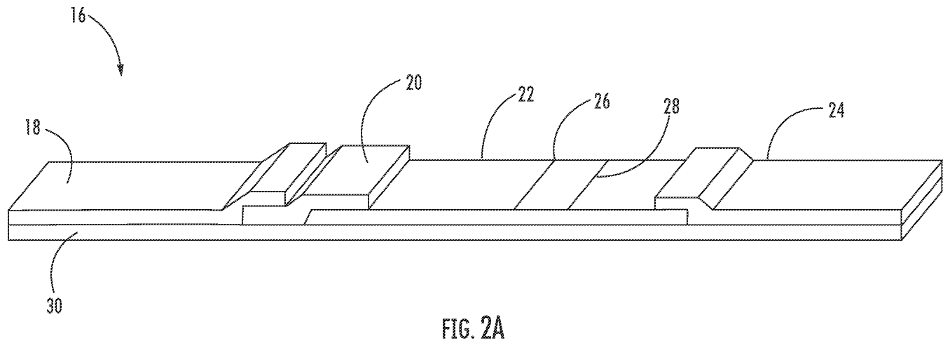

Referring to FIGS. 2A-2B, an illustrative embodiment of the nano-carbon immunochromatography strip 16 is shown. The nano-carbon immunochromatography strip 16 is shown having four sections or compartments: a sample application pad 18, a conjugate pad 20, a substrate membrane 22, and an adsorbent pad 24. In typical use, a sample to be analyzed is first applied to the sample application pad 18, which may be made of cellulose and/or glass fiber. Preferably, the sample application pad 18 is configured to transport the sample (or analyte) in a continuous manner to allow for proper sample components separation. The conjugate pad 20 may be made of glass fiber, cellulose, or polyester, and includes labeled biorecognition/detector molecules, such as antibodies 21 labeled with carbon nano-particles 23. The antibodies 21 are configured to bind to a specific component of the applied sample to form an analyte-antibody complex. The substrate membrane 22 may be a nitrocellulose membrane and include antibody capture lines, such as a test line 26 and a control line 28.

The test line 26 includes one or more primary antibodies 27 configured to bind to the analyte-antibody complex. The control line 28 includes one or more secondary antibodies 29 configured to bind to the analyte-antibody complex antibodies 21 labeled with carbon nano-particles 23. The adsorbent pad 24 acts like a wick and helps to maintain the flow rate of the liquid (sample) over the membrane. Each of the compartments, the sample application pad 18, the conjugate pad 20, the substrate membrane 22, and the adsorbent pad 24, may be fixed or mounted to a support structure or backing card 30. The support structure or backing card 30 may be made from a rigid or flexible material.

In a preferred embodiment, the systems, methods, and devices are designed to analyze a person's saliva for the presence of the COVID-19 virus. The systems, methods, and devices are designed to analyze a person's saliva for the presence of the COVID-19 virus rapidly, preferably within ten minutes or less, such as 5 minutes, one minute, or even less than one minute.

Saliva Sample

COVID-19 detection devices typically include a blood or throat swab, and the samples of antibody detection are whole blood or plasma. Studies by various medical institutions, see for example “Spit shines for easier coronavirus testing”, Science, Aug. 28, 2020, Vol 369, Issue 6607, pg 1041, have been done which indicate that salivary detection of new coronavirus may be more sensitive than using throat swabs. Use of a saliva test in accordance with embodiments of the invention may provide for the following advantages:

1) Saliva test is a non-invasive test, which is easy to obtain, and can even be collected by non-medical professional, i.e. by the user at home;

2) The sensitivity of a saliva sample may be higher than that of a throat swab; and

3) The results of a saliva test based on immunochromatography can be quickly obtained within 10 minutes, and no instrument or special or medical personnel is required.

The core technology of immunochromatographic saliva detection reagent, according to embodiments of the invention, is to solve the problems of saliva viscosity and slow chromatography speed.

### COVID-19 TEST EXAMPLE 1

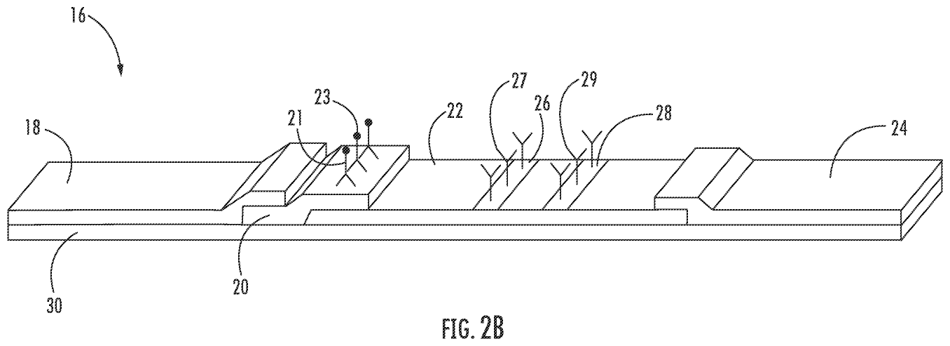

COVID-19 virus N or S protein was detected in human throat swabs, sputum and bronchoalveolar lavage fluid by double antibody sandwich. N and S protein antibodies labeled with carbon nanoparticles were used as indicator markers and dried on the glass fiber pad (conjugate pad 20). One end of the substrate membrane 22 (the nitrocellulose membrane, NC membrane) was connected with the sample application pad 18 and the other end of the NC membrane was connected with the absorbent pad 24. The detection line (T-line) 26 and quality control line (C-line) 28 on the NC membrane (the substrate membrane 22) were coated with N/S protein antibody and Goat anti-mouse IgG antibody, respectively. The nano-carbon immunochromatography device 10 may be configured so the T-line 26 detects the N or S protein (FIG. 2A) or to detect N-protein and S-protein separately, see FIG. 3, T-line 26A (N protein) and T-line 26B (S protein). While the detection antibody is described for detection of the N/S protein of the virus, other areas or portions/proteins or nucleic acids of the virus may be used as an antibody target. During the detection, the sample drops (saliva) are added to the nano-carbon immunochromatography device 10 (optionally through a sample hole), and the chromatography was carried out by capillary action. Through the conjugate pad 20 containing the nano-carbon conjugated monoclonal antibodies against N/S protein is rehydrated and reacts with nano-carbon antibody protein complex with carbon nanoparticles labeled N and S protein antibodies. Then, when the T-line 26 of the detection region with N/S protein antibody was immobilized, the microspheres labeled anti-N/S protein antibody protein antibody complex was formed, forming a black band on the detection line on the substrate membrane 22. Formation of the black band indicates a positive test result. Due to the excessive existence of the nano-carbon labeled antibody, no matter whether the sample contains N/S protein or not, the nano-carbon labeled antibody will be chromatographed to the C-line 28 to form a microsphere carbon labeled antibody Goat anti-mouse IgG complex. The quality control line 28 on the substrate membrane 22 will also form a black band.

The saliva detection reagent is designed as a bar sampling, the front end of which is a water absorbent rod with strong water absorption capacity, with the sample application pad 18 linked thereto. Finally, the sample passes through the substrate membrane 22 through chromatography to complete the detection.

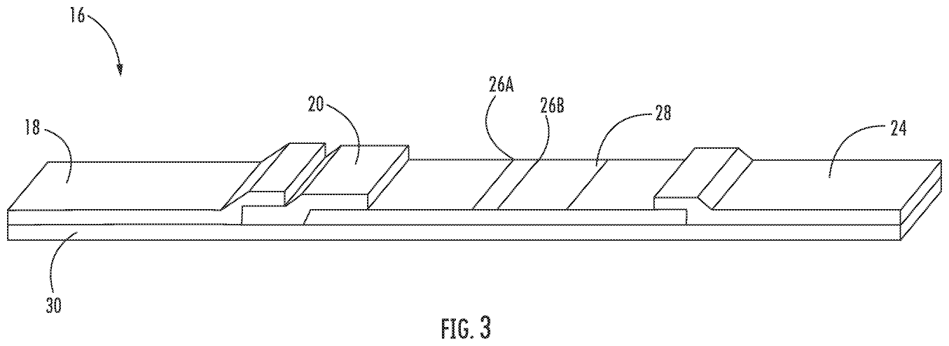

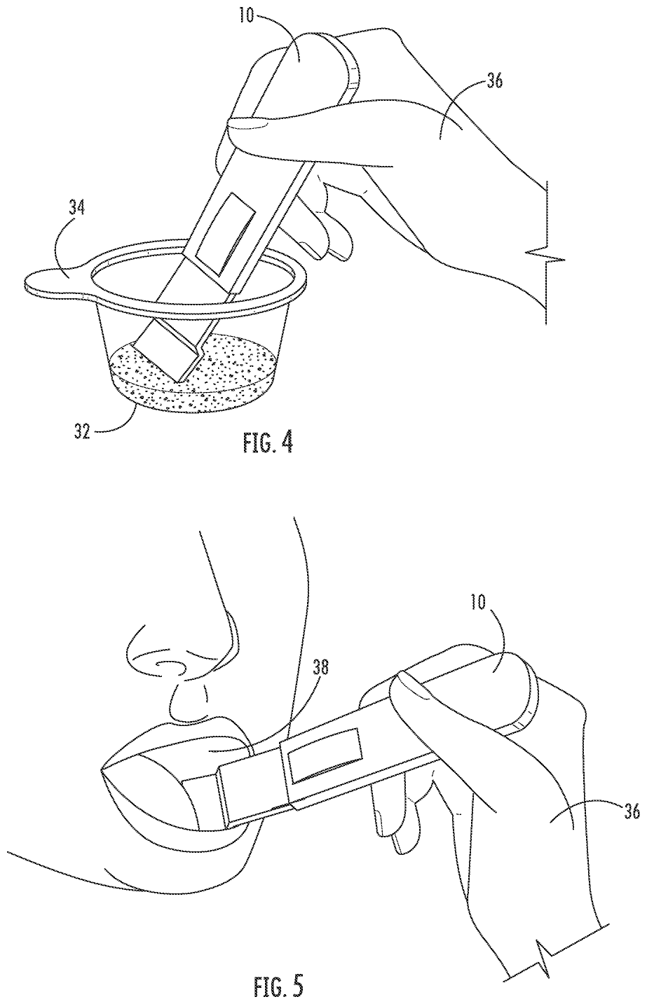

Referring to FIGS. 4 and 5, two illustrative sampling methods are shown. The first sampling method, see FIG. 4, includes collecting saliva 32 into the sample cup 34. Holding the nano-carbon immunochromatography device 10 in a user's hand, the nano-carbon immunochromatography device 10 may be immersed in the saliva 32. Alternatively, the nano-carbon immunochromatography device 10 may be placed directly into the user's mouth 38, see FIG. 5.

### TEST PREPARATION: EXAMPLES

Expression of N/S Protein of Recombinant Coronavirus

Construction of Expression Plasmid

a) PCR: primers were designed and the N/S protein gene sequence was amplified by PCT using positive serum as sample.

b) Ligation: the target fragment: carrier fragment 3:1, the plasmid is pET-28a (+) with 6× His tag, and ligase and buffer solution are added. After ligation at 16° C. or 23° C. for 1 hour, the appropriate target strain (DH5-α) was transfected.

c) Transfect bacteria: the transfected bacteria were coated on the plate, and the existence of the fragment was identified by colony PCR.

d) Strain storage: the positive colonies were cultured overnight at 37° C. and 200 rpm in a suitable LB medium. 0.5 ml overnight culture was extracted and mixed with 50% sterilized glycerin of the same volume. The positive colonies were stored at −80° C. and labeled.

Expression and Purification of Recombinant Protein

a) The strain BL21 (E. coli) containing N/S protein expression plasmid pET-28a was selected and inoculated into 100 ml LB medium containing 50 μg/ml kanamycin. The strain was shaken overnight.

b) 2 ml blank LB medium was taken as background, and the spectrophotometer was set as blank at 600 nm wavelength.

c) The overnight culture was poured into four bottles of 500 ml LB medium (100 ml), 50 mg/ml kanamycin was added to each bottle, and two bottles of 500 ml medium added with kanamycin and overnight culture were combined into one bottle. After shaking and mixing, the overnight culture medium was put back into two 500 ml bottles to ensure the homogeneity between the two bottles. The same was done for the other two bottles.

d) The culture was shaken for 2-3 hours, the OD value (600 nm) was determined, 12.5 ml of filtered and sterilized IPTG for every 500 ml was added, after each of two bottles are combined into one bottle, the filtered and sterilized IPTG of 100 mm was poured, mixed well, and then placed them back into two bottles of 500 ml to ensure the homogeneity between the two bottles. The same steps were performed for the other two bottles.

e) After induction of N/S protein expression, the cells were centrifuged.

f) The bacteria were mixed on ice with 200 ml crushing buffer (50 mm phosphoric acid buffer with pH 7.4) and ultrasonic crushing for 2 hours in ice bath was performed. Centrifugation and collection of supernatant was performed

g) The purified protein was eluted with imidazole and the eluent was collected.

The purity of eluate was identified by SDS-PAGE electrophoresis.

Making the Monoclonal Antibodies

a) Animal immunity: BALB/c mice were immunized with N/S protein recombinant antigen.

b) Cell fusion: spleen cells and SP2/0 cells of immunized mice were fused to obtain hybridoma cells.

c) Clone screening: the positive monoclonal hybridoma were screened by semi-solid medium by indirect ELISA method.

d) Monoclonal antibody identification: ELISA, antibody subclass identification kit and SDS-PAGE were used to determine the antibody titer, type and purity.

e) Antibody preparation: the monoclonal antibody was enriched by mouse ascites in vitro.

f) Antibody purification: ascites were purified by Protein A affinity column.

Preparation of Immunochromatographic Assay Kit

a) Nano-carbon dispersion: 100 μl/100 nm carbon spheres were added to 900 μl/5 mm borax buffer (pH 8.5), and subjected to ultrasound, three times.

b) Conjugate antibody: added 100 ug antibody and mixed in shaker for 1 hour.

c) Blocking: added 100 μL 10% BSA, mixed for 1 hour.

d) Washing: centrifugation was performed at 14000 RMP for 5 minutes, washing 4 times with 0.1M borax buffer (pH 8.5).

e) The labeled carbon nanoparticles were collected: 250 μl of 0.1 m borax buffer (pH 8.5, containing 1% BSA, 0.02% NaN₃), and then stored at 4° C.

Preparation of Conjugate Pad

a) The labeled carbon nanoparticles were ultrasonicated 5 times, and then evenly mixed. 37.5 μl was added into the diluent of 600 μl microspheres, 200 W ultrasound for 9 minutes on ice bath. After ultrasound, the mixture was added to 900 μl microsphere diluent to mix.

b) To a clean glass plate, the cut glass fiber kb50 fiber glass (3 cm×20 cm) was placed on the glass plate, and evenly coated the nano-carbon marker after ultrasonic dilution on the kb50 fiber glass

c) Dried at 37° C. overnight and stored in an aluminum foil bag.

Coating of Nitrocellulose Membrane/Substrate Membrane 22

The antigen and Goat and anti-mouse polyclonal antibodies were drawn on the nitrocellulose membrane (substrate membrane 22) as T-line 26 and C-line 28, respectively. The nitrocellulose membrane was coated with a dispenser and dried at 37° C. overnight.

Preparation of Sample Pad

In this experiment, 8964 glass fiber was selected as the sample pad. The sample pad was immersed in the buffer solution of sample pad, dried overnight at 37° C., and ready for use.

Assembly of Immunoassay Strip

Firstly, the nitrocellulose membrane is bonded to the backing light sheet, one end of the nitrocellulose membrane is bonded to the bonding pad and the sample pad, and the other end is bonded to the water absorbing pad. The adjacent components are overlapped by about 1 mm, and then the strip is cut off with a strip cutting machine, put into a plastic card or housing unit, and sealed in an aluminum foil bag. The immunoassay strip is ready for use as a single test application.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A rapid nano-carbon immunochromatography device for screening for presence of COVID-19 virus in saliva of an individual to be tested comprising:
a device configured to detect the presence of COVID virus from a saliva sample.

2. The rapid nano-carbon immunochromatography device according to claim 1, wherein said nano-carbon immunochromatography device configured to detect the presence of COVID virus from a saliva sample is a nano-carbon immunochromatography which includes carbon nanoparticles.

3. The rapid nano-carbon immunochromatography device according to claim 1, wherein said carbon nanoparticles are attached to antibodies configured to detect the presence of COVID-19 virus within said saliva sample.

4. The rapid nano-carbon immunochromatography device according to claim 1, wherein said nano-carbon immunochromatography device includes an indicator window configured to indicate the presence or absence of said COVID-19 virus within said saliva sample.

5. The rapid nano-carbon immunochromatography device according to claim 4, wherein said indicator window indicates the presence or absence of said COVID-19 virus within said saliva sample rapidly.

6. A rapid nano-carbon immunochromatography device for screening for presence of COVID-19 virus in saliva of an individual to be tested comprising:
an sample application pad configured for receiving or accepting a saliva sample to be analyzed for the presence or absence of said COVID-19 virus;
a conjugate pad, said conjugate paid comprising carbon nanoparticle labeled biorecognition molecules;
a substrate membrane; and
an adsorbent pad.

7. The rapid nano-carbon immunochromatography device for screening for presence of COVID-19 virus in saliva of an individual to be tested according to claim 6, wherein said carbon nanoparticle labeled biorecognition molecules are configured to bind to one or more components of said COVID-19 virus within said saliva sample applied to said sample application pad.

8. The rapid nano-carbon immunochromatography device for screening for presence of COVID-19 virus in saliva of an individual to be tested according to claim 6, wherein said substrate membrane first capture line having antibodies configured to bind to said nanoparticle labeled biorecognition molecules when said nanoparticle labeled biorecognition molecules are bound to a portion of said COVID-19 virus from said saliva sample.

9. The rapid nano-carbon immunochromatography device for screening for presence of COVID-19 virus in saliva of an individual to be tested according to claim 6, wherein said carbon nanoparticle labeled biorecognition molecules are carbon nanoparticle labeled antibodies configured to bind an N-antigen or S-antigen of said COVID-19 virus.

10. The rapid nano-carbon immunochromatography device for screening for presence of COVID-19 virus in saliva of an individual to be tested according to claim 6, wherein said detection of said COVID-19 virus is done in 10 minutes or less from application of said saliva to said application sample pad.

11. The rapid nano-carbon immunochromatography device for screening for presence of COVID-19 virus in saliva of an individual to be tested according to claim 6, further including a test line located on said substrate membrane.

12. The rapid nano-carbon immunochromatography device for screening for presence of COVID-19 virus in saliva of an individual to be tested according to claim 11, wherein said detection of said COVID-19 virus is indicated by formation of a black line along said test line.

13. The rapid nano-carbon immunochromatography device for screening for presence of COVID-19 virus in saliva of an individual to be tested according to claim 11, further including a control line located on said substrate membrane.

14. The rapid nano-carbon immunochromatography device for screening for presence of COVID-19 virus in saliva of an individual to be tested according to claim 11, wherein formation of a black line along said control line indicates presence of said carbon nanoparticle labeled biorecognition molecules.
